(19) United States
(12) Patent Application Publication
(10) Pub. No.: US 2014/0221836 A1
(43) Pub. Date: Aug. 7, 2014

(54) ULTRASOUND DIAGNOSTIC IMAGING APPARATUS

(71) Applicant: Konica Minolta, Inc., Tokyo (JP)

(72) Inventors: Osamu TAKEDA, Tokyo (JP); Ken INOUE, Tokyo (JP)

(73) Assignee: Konica Minolta, Inc., Tokyo (JP)

(21) Appl. No.: 14/171,726

(22) Filed: Feb. 3, 2014

(30) Foreign Application Priority Data
Feb. 5, 2013 (JP) 2013-020000

(51) Int. Cl. A61B 8/00

(52) U.S. Cl. CPC A61B 8/461
USPC 600/443

(57) ABSTRACT

An ultrasound diagnostic imaging apparatus includes an ultrasound probe, a display unit, a touch panel and a control unit. The touch panel is disposed in such a way as to be superposed on a display screen of the display unit. The control unit (i) displays a body mark representing a body part of a subject on the display screen, (ii) sets an operation receivable region on the touch panel in such a way as to be suitable for the displayed body mark, (iii) detects a touched point in the operation receivable region and (iv) performs display position setting to control the display unit to display a probe mark representing the ultrasound probe in form according to the detected touched point.

### FIELD OF THE INVENTION

The present invention relates to an ultrasound diagnostic imaging apparatus.

### DESCRIPTION OF THE RELATED ART

An ultrasound diagnostic imaging apparatus has been used as a medical diagnostic imaging apparatus to carry out tests (ultrasound examinations) of biological tissues of the heart, arms, legs, breasts and the like. In practice, when one test is carried out, ultrasound image data of multiple body parts are obtained through the test. The ultrasound image data thus obtained are stored in a storage medium or the like for future diagnosis. An interpreter such as a doctor selects ultrasound image data of a body part which is a target for diagnosis from the stored ultrasound image data, displays an ultrasound image based thereon on a display device or the like and makes a diagnosis on the basis of the displayed ultrasound image.

It is necessary for an interpreter to be able to select ultrasound image data of a desired body part. Hence, in a conventional ultrasound diagnostic imaging apparatus, symbol image data called a body mark is combined and stored with ultrasound image data for storage. Ultrasound image data combined with a body mark makes it easy for an interpreter to visually recognize a body part which is a target for diagnosis and accordingly is useful in selecting ultrasound image data.

It is important to show on which point of a body part a scan is performed to obtain an ultrasound image as well as displaying a body. mark. Hence, a probe mark which represents an ultrasound probe to transmit/receive ultrasound is put on a body mark. The probe mark is set at a certain point on the body mark by operating controllers such as a trackball and switches disposed on an operation panel, which is described, for example, in Japanese Patent Application Laid-Open Publication No. 2005-40301.

### BRIEF SUMMARY OF THE INVENTION

However, in the ultrasound diagnostic imaging apparatus described in Japanese Patent Application Laid-Open Publication No. 2005-40301, because various controllers and switches are needed to be operated insetting the positions of the body mark and the probe mark, the number of operations therefore is large, and a movement distance of a hand to perform the operations is long. Accordingly, there are problems of very complicated operation and low test efficiency.

The present invention has been conceived in view of the above-described circumstances, and objects include providing an ultrasound diagnostic imaging apparatus having improved usability in inputting a body mark.

In order to achieve at least one of the objects, according to an aspect of the present invention, there is provided an ultrasound diagnostic imaging apparatus including: an ultrasound probe which outputs transmissive ultrasound to a subject and receives reflected ultrasound from the subject to obtain a reception signal; a display unit including a display screen on which an ultrasound image based on ultrasound image data generated on the basis of the reception signal obtained by the ultrasound probe is displayed; a touch panel disposed in such a way as to be superposed on the display screen of the display unit; and a control unit which (i) displays a body mark representing a body part of the subject on the display screen of the display unit, (ii) sets an operation receivable region on the touch panel in such a way as to be suitable for the displayed body mark, (iii) detects a touched point in the operation receivable region and (iv) performs display position setting to control the display unit to display a probe mark representing the ultrasound probe in form according to the detected touched point in the operation receivable region.

Preferably, in the ultrasound diagnostic imaging apparatus, the operation receivable region includes an effective operation region set in such a way as to be superposed on the displayed body mark, and in the display position setting, the control unit controls the display unit to display the probe mark at the detected touched point in the effective operation region.

Preferably, in the ultrasound diagnostic imaging apparatus, the operation receivable region includes an angle setting operation region set in such a way as to be superposed on an angle setting guide displayed around the body mark, and in the display position setting, the control unit controls the display unit to display the probe mark at a display angle corresponding to the detected touched point in the angle setting operation region.

Preferably, in the ultrasound diagnostic imaging apparatus, the control unit (i) sets a set operation region in such a way as to be superposed on a set button displayed near the body mark and (ii) in the display position setting, performs control to complete the display position setting when detecting a touch on the set operation region.

Preferably, in the ultrasound diagnostic imaging apparatus, in the display position setting, the control unit controls the display unit to display the body mark larger than the body mark which is displayed with the ultrasound image when the display position setting is completed.

Preferably, in the ultrasound diagnostic imaging apparatus, in the display position setting, the control unit performs control to complete the display position setting when detecting no touch on the touch panel for a predetermined time.

Preferably, in the ultrasound diagnostic imaging apparatus, the control unit (i) sets a reset operation region on the touch panel in such a way as to be superposed on the body mark which is displayed with the ultrasound image when the display position setting is completed and (ii) performs the display position setting when detecting a touch on the reset operation region.

### BRIEF DESCRIPTION OF THE SEVERAL VIEWS OF THE DRAWING

The present invention is fully understood from the detailed description given hereinafter and the accompanying drawings, which are given by way of illustration only and thus are not intended to limit the present invention, wherein:

FIG. 1 is a block diagram of a medical image management system according to an embodiment of the present invention;

FIG. 2 shows arrangement of apparatuses in a medical facility where the medial image management system according to the embodiment is used;

FIG. 3 shows the external appearance of an ultrasound diagnostic imaging apparatus;

FIG. 4 is a block diagram showing a functional configuration of the ultrasound diagnostic imaging apparatus;

FIG. 5 is a flowchart of image file generation processing;

FIG. 6 is a flowchart of patient information input processing;

FIG. 7 is a flowchart of body mark input processing;

FIG. 8 illustrates an ultrasound diagnostic screen;

FIG. 9 illustrates a patient registration screen;

FIG. 10 illustrates the patient registration screen;

FIG. 11 illustrates the patient registration screen;

FIG. 12 illustrates the patient registration screen;

FIG. 13 illustrates the ultrasound diagnostic screen;

FIG. 14 illustrates the ultrasound diagnostic screen;

FIG. 15 illustrates the ultrasound diagnostic screen;

FIG. 16 illustrates the ultrasound diagnostic screen;

FIG. 17 illustrates the ultrasound diagnostic screen;

FIG. 18 illustrates the ultrasound diagnostic screen;

FIG. 19 illustrates the ultrasound diagnostic screen;

FIG. 20 illustrates the ultrasound diagnostic screen;

FIG. 21 illustrates the ultrasound diagnostic screen;

FIG. 22 illustrates the ultrasound diagnostic screen;

FIG. 23 illustrates the ultrasound diagnostic screen;

FIG. 24 illustrates the ultrasound diagnostic screen; and

FIG. 25 shows another arrangement of the ultrasound diagnostic imaging apparatuses in a medical facility.

### DETAILED DESCRIPTION OF THE INVENTION

In the following, a medical image management system according to an embodiment of the present invention is described with reference to the drawings. However, the scope of the present invention is not limited to the illustrated embodiment. In the following, components having the same function and configuration are provided with the same reference number, and description thereof is not repeated.

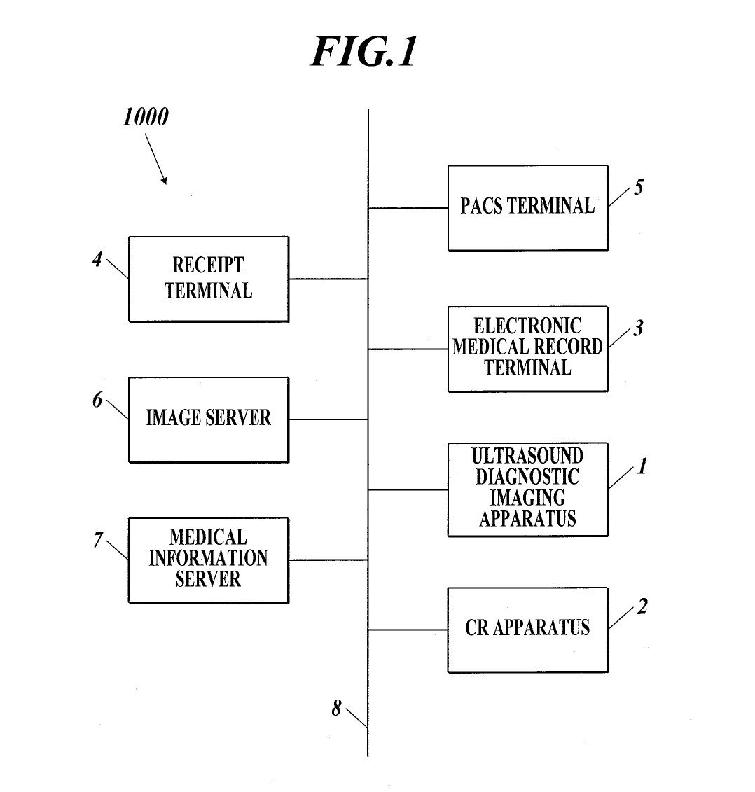

As shown in FIG. 1, a medical image management system 1000 includes an ultrasound diagnostic imaging apparatus 1, a CR (Computed Radiography) apparatus 2, an electronic medical record (karte) terminal 3, a receipt (rezept) terminal 4, a PACS (Picture Archiving and Communication System) terminal 5, an image server 6 and a medical information server 7. These apparatuses and so forth are connected to each other, for example, via a LAN (Local Area Network) 8.

In general, as a communication system in a medical facility, DICOM (Digital Imaging and COmmunication in Medicine) standard is used, and for communication between apparatuses connected via a LAN, DICOM MWM (DICOM Modality Worklist Management) or DICOM MPPS (DICOM Modality Performed Procedure Step) is used. The communication system applicable to the embodiment is not limited thereto.

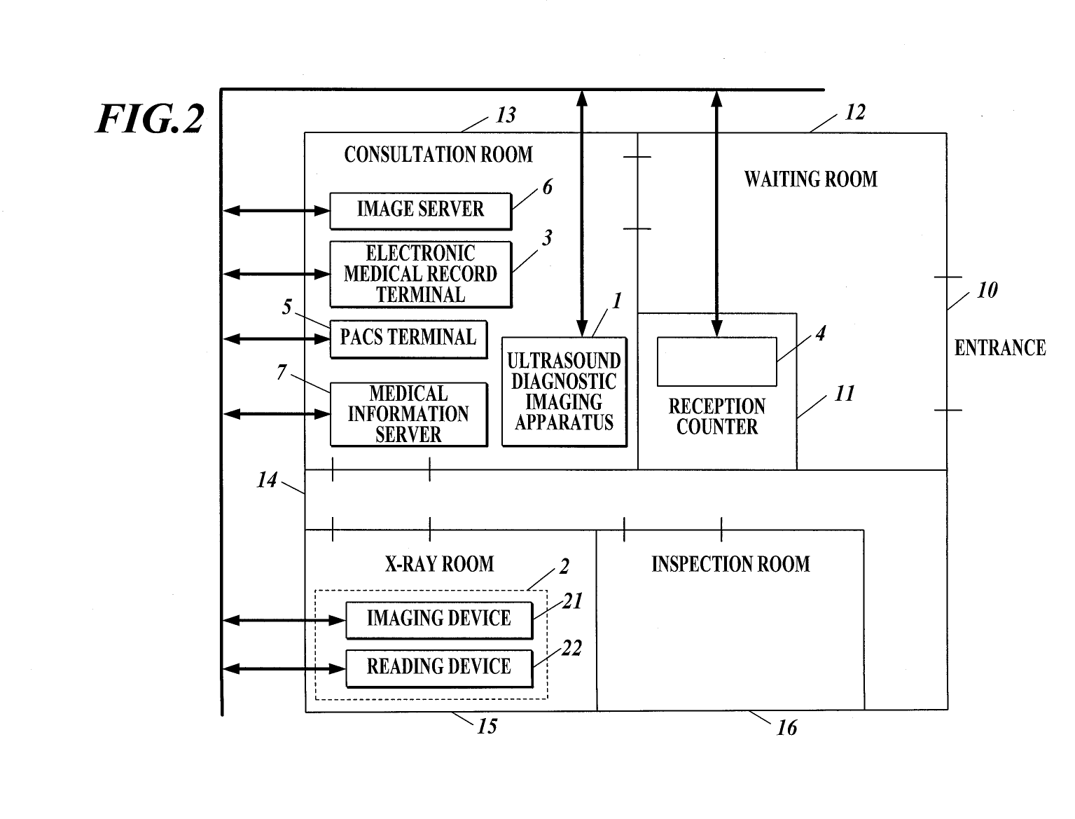

For example, in a small-sized medical facility such as a private doctor's office or a clinic, the apparatuses are arranged, for example, as shown in FIG. 2.

That is, through an entrance 10, there are a reception counter 11 to accept patients and a waiting room 12. At the reception counter 11, a receptionist is stationed. The receptionist gives receipt tickets with receipt numbers to recognize individual patients (for example, sequential numbers in the order of acceptance of patients in one day) printed to patients who come to the medical facility in the order of acceptance of the patients. On the reception counter 11, the receipt terminal 4 to calculate insurance points, bills and so forth is placed. The receptionist refers to a consultation card or the like which a patient has and inputs a receipt number, a patient name and a patient ID of the patient into the receipt terminal 4 to be correlated with each other. The receipt terminal 4 requests the medical information server 7 to send an electronic medical record (karte) as medical record data in which detailed information about the patient who matches the input patient name and patient ID is described. After the receipt terminal 4 receives the electronic medical record from the medical information server 7, the receptionist checks the content of the electronic medical record on a display screen of the receipt terminal 4 and commands the receipt terminal 4 to send the electronic medical record to the electronic medical record terminal 3. Then, the receipt terminal 4 sends the electronic medical record of the patient, who has come to the medical facility, to the electronic medical record terminal 3. After the patient is examined, the receptionist inputs necessary information relating to a bill and a rezept (an itemized statement of medical expenses) (bill calculation and insurance point calculation) into the receipt terminal 4 on the basis of information described in the electronic medical record. The receptionist checks a doctor's fee which is displayed on the display screen of the receipt terminal 4 when input of the information relating to the rezept is completed, receives the fee from the patient and issues and gives a receipt for the fee to the patient.

A consultation room 13 where a doctor examines a patient, makes a diagnosis and so forth is next to the waiting room 12 via a door or the like. The electronic medical record terminal 3 and the PACS terminal 5 are placed on a not-shown desk used for consultation in the consultation room 13, and the image server 6, the medical information terminal 7 and the ultrasound diagnostic imaging apparatus 1 are arranged at predetermined positions in the consultation room 13, for example. In the electronic medical record terminal 3, names of patients for consultation are stored in the order of reception of electronic medical records from the receipt terminal 4. Thus, the patients are on the waiting list. The patients wait for their turns in the waiting room 12. When a doctor performs an update operation of the patients for consultation, the electronic medical record terminal 3 displays the content of an electronic medical record for the next patient on a display screen thereof. Then, the doctor lets the patient enter the consultation room 13 and starts examination. The doctor examines the patient and inputs the content of the examination into the electronic medical record terminal 3. The doctor carries out a test (an ultrasound examination) with the ultrasound diagnostic imaging apparatus 1 as needed. The ultrasound diagnostic imaging apparatus 1 stores ultrasound image data obtained by the test into the image server 6. The image server 6 has an image DB (DataBase) and stores therein image data such as the ultrasound image data. The image data stored in the image server 6 is read by the PACS terminal 5, and a medical image based thereon is displayed on a display screen of the PACS terminal 5. The doctor explains the result of the test and so forth to the patient, using the medical image displayed on the display screen of the PACS terminal 5 and also inputs findings into the electronic medical record terminal 3. When the doctor finishes examining the patient and inputs an examination end command into the electronic medical record terminal 3, the electronic medical record terminal 3 sends the electronic medical record to the receipt terminal 4. The patient leaves the consultation room 13 and returns to the waiting room 12. The doctor performs an update operation of the patients for consultation as described above and lets the next patient enter the consultation room 13 and examines the patient.

The consultation room 13 faces, across a passage 14, an X-ray room 15 where X-ray photography is carried out. In the X-ray room 15, the CR apparatus 2 composed of an imaging device 21 and a reading device 22 is arranged. Next to the X-ray room 15, there is an inspection room 16.

Next, the ultrasound diagnostic imaging apparatus 1 of the embodiment is described.

The ultrasound diagnostic imaging apparatus 1 is an apparatus to display/output condition of biological tissues of a patient (i.e. a subject) as an ultrasound image(s). More specifically, the ultrasound diagnostic imaging apparatus 1 transmits ultrasound (transmissive ultrasound) into a subject such as a living body and receives reflected waves (reflected ultrasound or echo) of the ultrasound reflected inside the subject. The ultrasound diagnostic imaging apparatus 1 converts the received reflected ultrasound into electric signals and generates ultrasound image data on the basis of the electric signals. The ultrasound diagnostic imaging apparatus 1 displays the internal condition of the subject as ultrasound images on the basis of the generated ultrasound image data. In addition, the ultrasound diagnostic imaging apparatus 1 generates supplementary information relating to the generated ultrasound image data on the basis of patient information which is information about the patient (subject). The ultrasound diagnostic imaging apparatus 1 attaches the supplementary information to the ultrasound image data to generate an image file composed of DICOM image data in accordance with the DICOM standard.

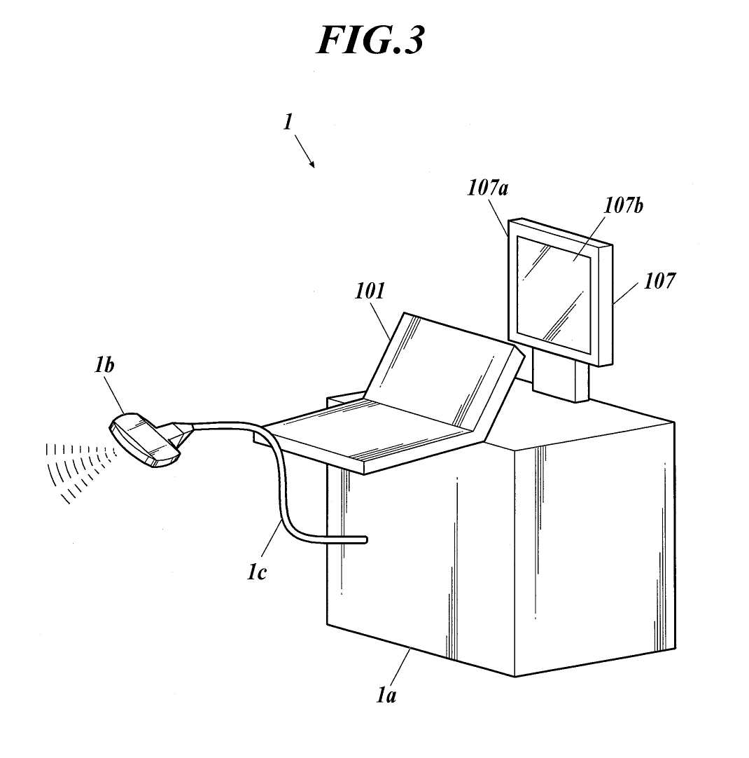

As shown in FIG. 3, the ultrasound diagnostic imaging apparatus 1 includes a main body 1a and an ultrasound probe 1b. The ultrasound probe 1b transmits the transmissive ultrasound into a subject and receives the reflected ultrasound from the inside of the subject. The main body 1a is connected to the ultrasound probe 1b via a cable is and transmits drive signals composed of electric signals to the ultrasound probe 1b to make the ultrasound probe 1b transmit the transmissive ultrasound into a subject. The main body 1a also (i) receives reception signals composed of electric signals which the ultrasound probe 1b generates when receiving the reflected ultrasound from the inside of the subject and (ii) generates ultrasound image data as described above.

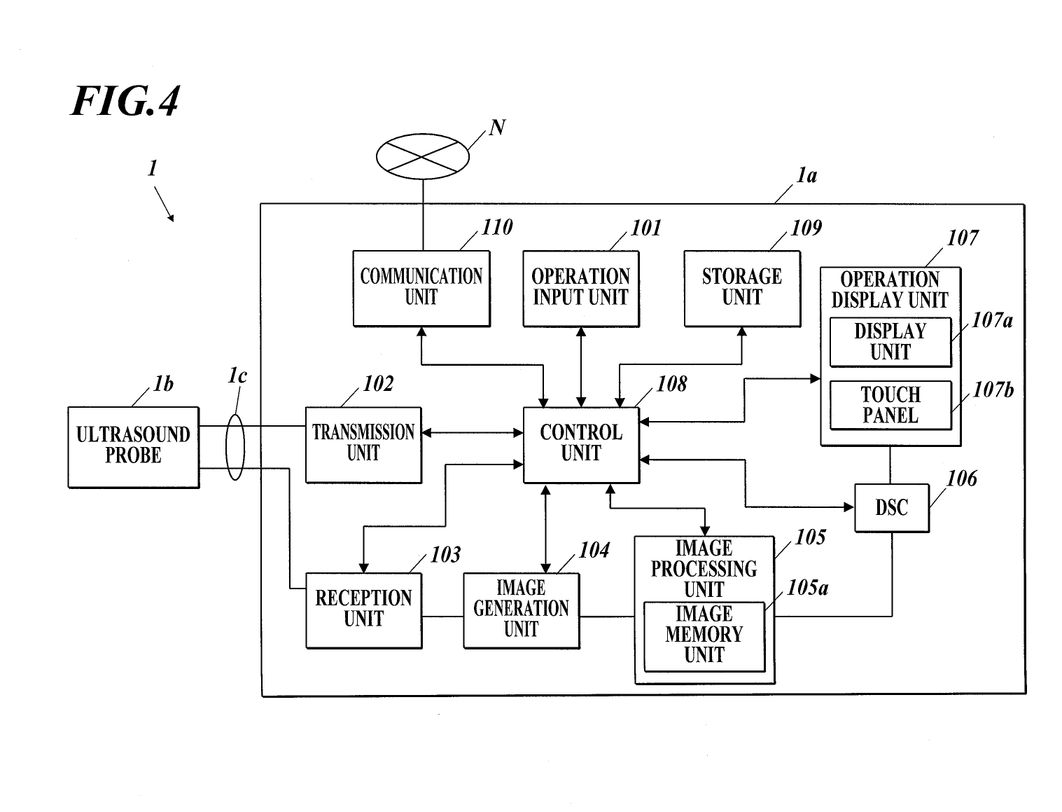

As shown in FIG. 4, the main body 1a includes, for example, an operation input unit 101, a transmission unit 102, a reception unit 103, an image generation unit 104, an image processing unit 105, a DSC (Digital Scan Converter) 106, an operation display unit 107, a control unit 108, a storage unit 109 and a communication unit 110.

The operation input unit 101 includes, for example, various switches, buttons, a trackball, a mouse and a keyboard to input, for example, commands to start diagnosis and data such as personal information about subjects and outputs operation signals to the control unit 108.

The transmission unit 102 is a circuit to supply the drive signals, which are composed of electric signals, to the ultrasound probe 1b via the cable 1c under control of the control unit 108 to make the ultrasound probe 1b generate the transmissive ultrasound. The transmission unit 102 includes, for example, a clock generation circuit, a delay circuit and a pulse generation circuit. The clock generation circuit is a circuit to generate clock signals which determine transmission timings of the drive signals and the transmission frequency thereof. The delay circuit is a circuit to individually set delay times with respect to respective paths for transducers, the delay times by which the transmission timings of the drive signals are delayed, and to delay transmission of the drive signals by the set delay times to converge transmission beams composed of the transmissive ultrasound. The pulse generation circuit is a circuit to generate pulse signals as the drive signals at predetermined intervals. The transmission unit 102 thus configured drives some continuous transducers (for example, 64 transducers) among a plurality of transducers (for example, 192 transducers) arranged in the ultrasound probe 1b so that the transmissive ultrasound is generated. The transmission unit 102 changes transducers to drive in an orientation direction each time the transmissive ultrasound is generated so that a scan is performed.

The reception unit 103 is a circuit to receive the reception signals, which are composed of electric signals, from the ultrasound probe 1b via the cable 1c under control of the control unit 108. The reception unit 103 includes, for example, an amplifier, an A/D converter circuit and a phasing addition circuit. The amplifier is a circuit to amplify the reception signals at amplification factors individually preset with respect to the respective paths for the transducers. The A/D converter circuit is a circuit to perform A/D conversion on the amplified reception signals. The phasing addition circuit is a circuit to give the delay times, which are individually set with respect to the respective paths for the transducers, to the A/D conversion-performed reception signals to phase the signals and to add up these signals (phasing addition) to generate sound ray data.

The image generation unit 104 performs envelope detection, logarithmic amplification or the like on the sound ray data generated by the reception unit 103 and adjusts dynamic range or gain to perform brightness conversion, thereby generating B mode image data. That is, the B mode image data expresses strength of the reception signals by brightness. The image generation unit 104 may be configured to generate A mode image data, M mode image data and/or image data by Doppler method besides the B mode image data.

The image processing unit 105 includes an image memory unit 105a composed of a semiconductor memory such as a DRAM (Dynamic Random Access Memory). The image processing unit 105 stores the B mode image data, which are output from the image generation unit 104, in the image memory unit 105a frame by frame. The image data frame by frame may be referred to as ultrasound image data or frame image data. The frame image data stored in the image memory unit 105a is sent to the DSC 106 under control of the control unit 108.

The DSC 106 converts the frame image data received from the image processing unit 105 into image signals in accordance with a television signal scanning mode and outputs the image signals to the operation display unit 107.

The operation display unit 107 includes a display unit 107a and a touch panel 107b.

As the display unit 107a, a display device such as an LCD (Liquid Crystal Display), a CRT (Cathode-Ray Tube) display, an organic EL (Electronic Luminescence) display, an inorganic EL display or a plasma display can be used. The display unit 107a displays images on a display screen thereof in response to the image signals output from the DSC 106.

The touch panel 107b is a pressure-sensitive (resistance-film type) touch panel which is composed of transparent electrodes arranged in a grid pattern and is formed on the display screen of the display unit 107a. The touch panel 107b detects X coordinates and Y coordinates of points pressed on a screen thereof with a finger as voltage values and outputs positional signals corresponding to the detected voltage values as operation signals to the control unit 108. The touch panel 107b is not limited to the pressure sensitive type, and an appropriate type can be selected from among various types such as the electrostatic capacity type.

The control unit 108 includes, for example, a CPU (Central Processing Unit), a ROM (Read Only Memory) and a RAM (Random Access Memory) and reads a system program and a program(s) of various processing programs stored in the ROM, opens the read programs in the RAM and performs centralized control of actions of the components of the ultrasound diagnostic imaging apparatus 1 in accordance with the opened programs.

The ROM is composed, for example, of a nonvolatile memory such as a semiconductor memory and stores therein, for example, the system program for the ultrasound diagnostic imaging apparatus 1; the various processing programs executable on the system program to perform, for example, image file generation processing, patient information input processing and body mark input processing, which are described below; and various data such as a gamma table. These programs are stored in a program code readable by a computer, and the CPU acts step by step in accordance with the program code.

The RAM forms a work area where the various programs executed by the CPU and data relevant to the programs are temporarily stored.

The storage unit 109 is composed, for example, of a mass storage medium such as an HDD (Hard Disk Drive) and stores therein, for example, the ultrasound image data generated by the image processing unit 105.

The communication unit 110 includes a LAN adapter and performs data transmission/reception with the apparatuses connected via a network N such as the LAN 8.

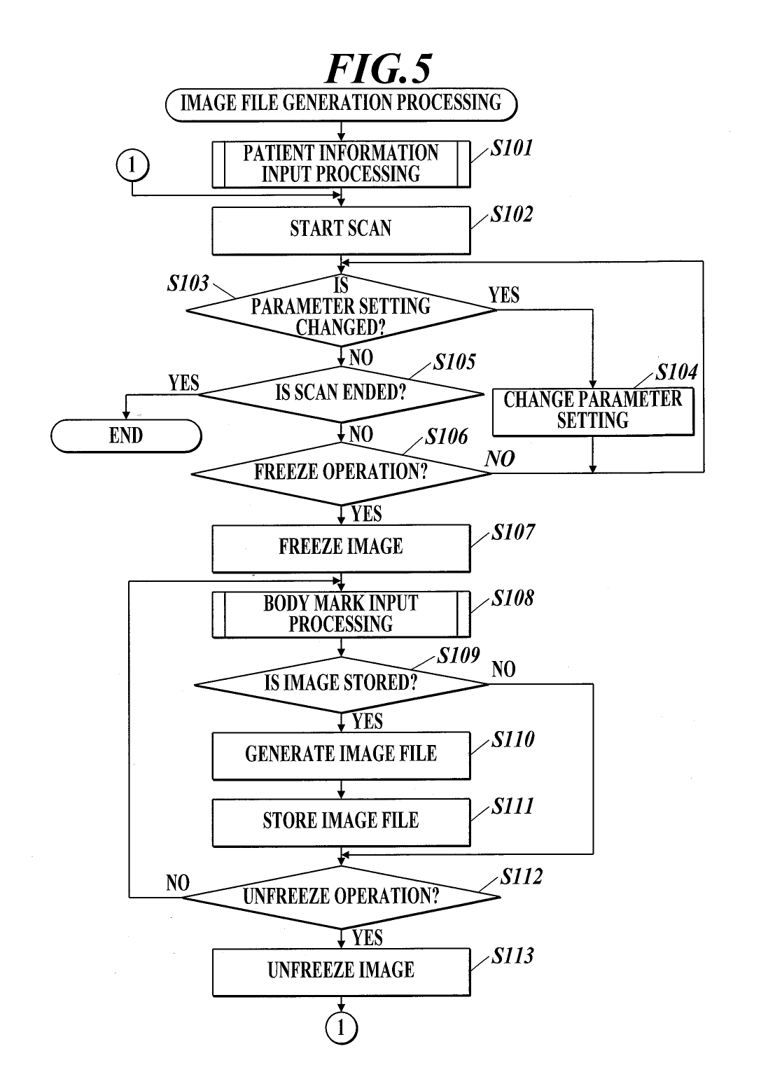

Next, the image file generation processing performed by the control unit 108 of the ultrasound diagnostic imaging apparatus 1 of the medical image management system 1000 configured as described above is described with reference to FIG. 5. The image file generation processing is performed each time a test (ultrasound examination) is carried out by the ultrasound diagnostic imaging apparatus 1. For example, the image file generation processing is performed in response to a predetermined test execution operation by an operator such as a doctor or a technician.

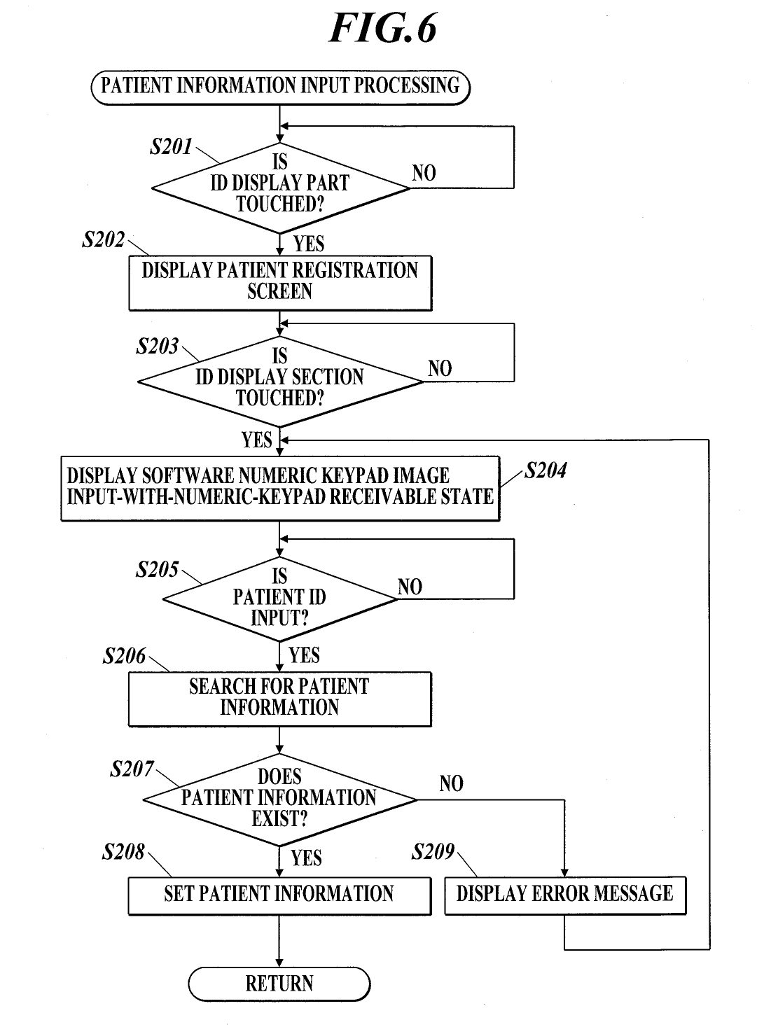

First, the control unit 108 performs the patient information input processing (Step S101). Through the patient information input processing, information such as a patient name, a patient ID and the like can be registered before a test is carried out. The registered information is correlated and stored with ultrasound image data, so that when an ultrasound image based thereon is referred to after the ultrasound image data is obtained, it can be recognized whose ultrasound image it is. Now, the patient information input processing is described with reference to FIG. 6.

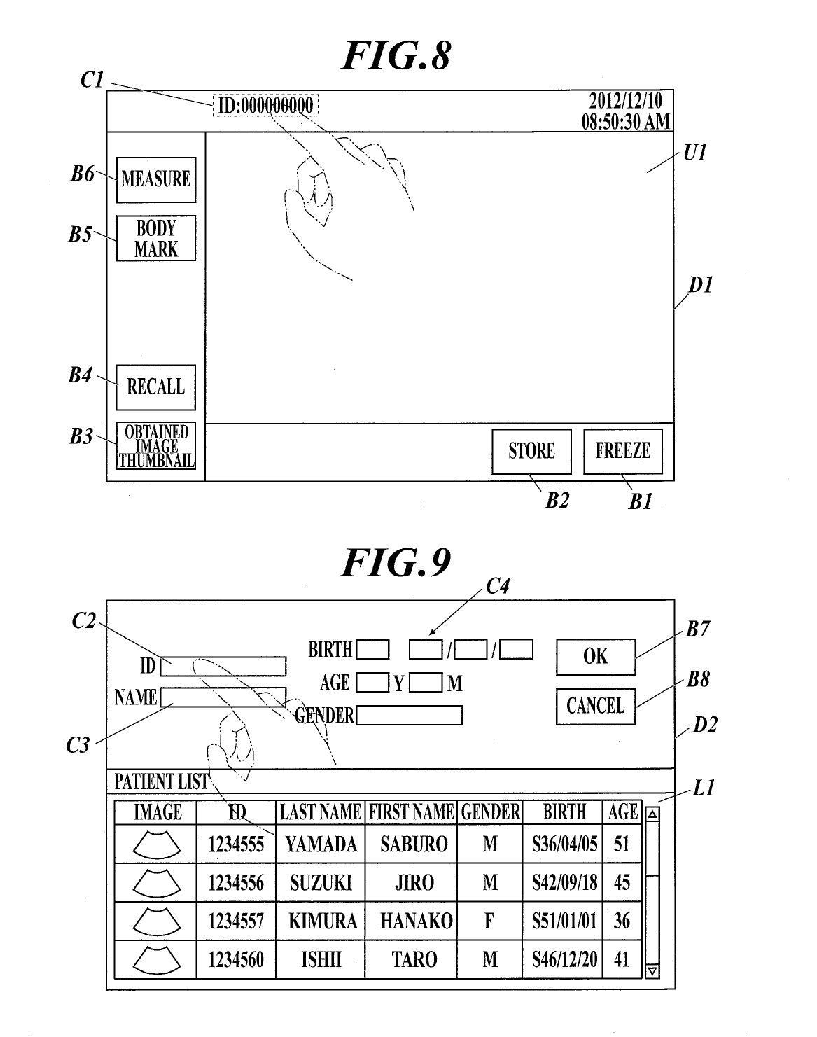

First, the control unit 108 determines whether or not a touch operation is performed on an ID display part displayed on the display screen of the display unit 107a (Step S201). More specifically, when the patient information input processing is performed, an ultrasound diagnostic screen D1 shown in FIG. 8 is displayed on the display screen of the display unit 107a. On the ultrasound diagnostic screen D1, an ultrasound image display region U1 is arranged, and around the ultrasound image display region U1, images of a freeze button B1, a store button B2, an obtained image thumbnail button B3, a recall button B4, a body mark button B5 and a measure button B6 are displayed. The functions of these buttons are described below. The types of buttons arranged on the ultrasound diagnostic screen D1 are not limited thereto. Above the ultrasound image display region U1, an ID display part C1 is arranged. When a touch operation is performed on any of the images of the buttons B1 to B6 and the ID display part C1, the touch panel 107b detects the touch operation and outputs an operation signal corresponding to the touched point (image) to the control unit 108. At Step S201, the control unit 108 determines whether or not a touch operation is performed on the ID display part C1 as shown in FIG. 8 on the ultrasound diagnostic screen D1 configured as described above.

When determining that no touch operation is performed on the ID display part C1 (Step S201; NO), the control unit 108 repeats Step S201 until determining that a touch operation is performed on the ID display part C1. On the other hand, when determining that a touch operation is performed on the ID display part C1 (Step S201; YES), the control unit 108 displays a patient registration screen D2 shown in FIG. 9 on the display screen of the display unit 107a (Step S202). On the patient registration screen D2, a patient list L1 is arranged at the lower part, an ID display section C2, a patient name display section C3 and a patient information display part C4 are arranged at the upper part, and images of an OK button B7 and a cancel button B8 are arranged on the right of the patient information display part C4. In the patient list L1, all the patients having undergone tests with the ultrasound diagnostic imaging apparatus 1 are listed. The information about the patients (patient information) listed in the patient list L1 is stored, for example, in the storage unit 109. In the patient list L1, with respect to each patient, the patient ID, name, gender, birthday, age and a thumbnail of the most-recently-obtained ultrasound image are displayed. By displaying the thumbnail, an examiner such as a doctor can recognize with one look for which body part of the patient ultrasound imaging has been performed at the most recent test and accordingly can easily know the result of the past test of the patient. In the patient list L1 shown in FIG. 9, only some patients' information is displayed. However, for example, by performing a touch operation on a scroll bar displayed on the right on the patient list L1, the patient list L1 can be scrolled. Thus, the other patients' information can be displayed. In the ID display section C2, the ID of a patient selected as described below is displayed. In the patient name display section C3, the name of the selected patient is displayed. In the patient information display part C4, the birthday, age and gender of the selected patient are displayed. If detecting a touch operation on the cancel button B8, the control unit 108 ends the patient information input processing without registering the patient and changes the display screen of the display unit 107a from the patient registration screen D2 to the ultrasound diagnostic screen D1 shown in FIG. 8.

Next, the control unit 108 determines whether or not a touch operation is performed on the ID display section C2 (Step S203). More specifically, the control unit 108 determines whether or not a touch operation is performed on the ID display section C2 as shown in FIG. 9 on the patient registration screen D2.

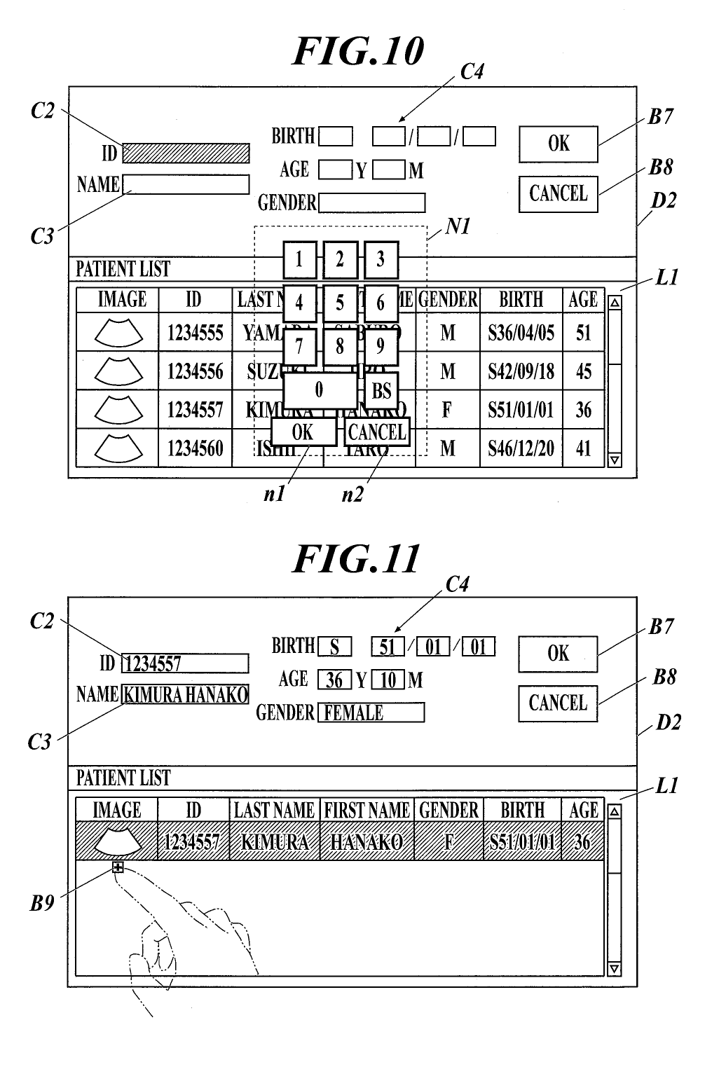

When determining that no touch operation is performed on the ID display section C2 (Step S203; NO), the control unit 108 repeats Step S203 until determining that a touch operation is performed on the ID display section C2. On the other hand, when determining that a touch operation is performed on the ID display section C2 (Step S203; YES), as shown in FIG. 10, the control unit 108 displays a software numeric keypad N1 over the center of the patient registration screen D2 and receives a touch operation on the software numeric keypad N1 (Step S204). Each time a touch operation is performed on the software numeric keypad N1, the same number as that of a key on which the touch operation is performed is displayed in the ID display section C2 to form a sequence of numbers. Thus, a patient ID can be input.

In the embodiment, when a touch operation is performed on the ID display section C2, the software numeric keypad N1 is displayed. However, the software numeric keypad N1 may be displayed when the display screen is changed from the ultrasound diagnostic screen D1 to the patient registration screen D2. Such action may be performed by a configuration.

The control unit 108 determines whether or not input of a patient ID is completed (Step S205). More specifically, the control unit 108 determines that input of a patient ID is completed when detecting a touch operation on an OK button n1 after input of the patient ID is performed with the software numeric keypad N1 as described above. If detecting a touch operation on a cancel button n2, the control unit 108 cancels the input patient ID and hides the software numeric keypad N1.

When determining that input of a patient ID is not completed (Step S205; NO), the control unit 108 repeats Step S205 until determining that input of a patient ID is completed. On the other hand, when determining that input of a patient ID is completed (Step S205; YES), the control unit 108 searches the storage unit 109 for patient information agreeing with the input patient ID (Step S206).

The control unit 108 determines whether or not the patient information agreeing with the input patient ID is stored in the storage unit 109 on the basis of the search result of the patient information (Step S207).

When determining that the patient information agreeing with the input patient ID is stored in the storage unit 109 (Step S207; YES), the control unit 108 reads the patient information from the storage unit 109 and sets the patient information (Step S208) and then ends the patient information input processing. That is, the control unit 108 displays the patient information, for example, as shown in FIG. 11. More specifically, on the patient registration screen D2, the control unit 108 displays only the patient information agreeing with the input patient ID in the patient list L1, displays the input patient ID in the ID display section C2, displays the patient name correlated with the input patient ID in the patient name display section C3 and displays the birthday, age and gender of the patient correlated with the input patient ID in the patient information display part C4. When detecting a touch operation on the OK button B7 after reading the patient information from the storage unit 109 as described above, the control unit 108 changes the display screen to the ultrasound diagnostic screen D1 shown in FIG. 8. At the time, in the ID display part C1, the input patient ID is displayed. On the other hand, when determining that the patient information agreeing with the input patient ID is not stored in the storage unit 109 (Step S207; NO), the control unit 108 displays a predetermined error message (Step S209) and moves to Step S204.

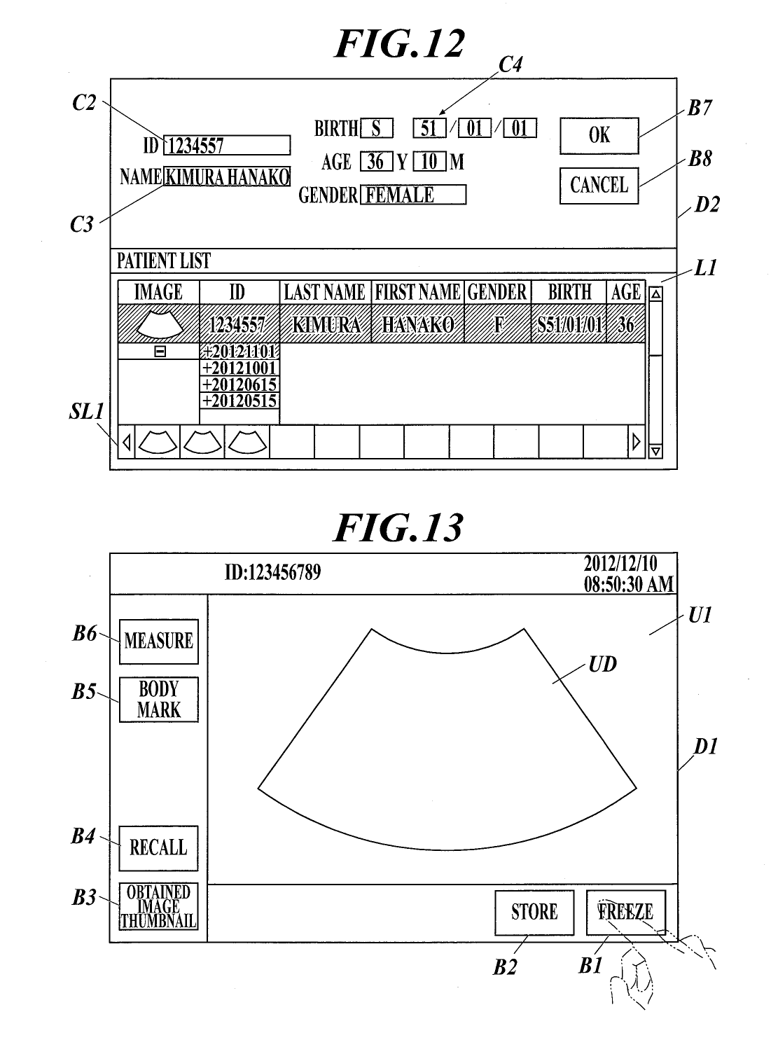

In the embodiment, when a touch operation is performed on an extension button B9 displayed, for example, under the patient information displayed in the patient list L1 as shown in FIG. 11, dates of the past tests with the ultrasound diagnostic imaging apparatus 1 are displayed in a list form as shown in FIG. 12. When a touch operation is performed on one of the listed dates, thumbnails based on ultrasound image data stored on the date are displayed in a thumbnail list SL1. Consequently, it can be easily known what kind of ultrasound imaging the patient has undergone at the past tests while the patient information is registered on the patient registration screen D2.

Further, in the embodiment, it is possible that when a patient ID is input, a command to search for patient information agreeing with the input patient ID is sent to the electric medical record terminal 3 or the medical information server 7 connected to the ultrasound diagnostic imaging apparatus 1 via the LAN 8, and if the patient information is stored in the electric medical record terminal 3 or the medical information server 7, the patient information is sent to the ultrasound diagnostic imaging apparatus 1 so that the ultrasound diagnostic imaging apparatus 1 obtains the patient information.

Further, in the embodiment, it is possible that a hard copy of an electric medical record displayed on the display screen of the electric medical record terminal 3 or the like is obtained, and the content of the hard copy is analyzed with a function such as an OCR (Optical Character Reader) to extract patient information therefrom. For example, at a private doctor's office, a doctor carries out a text with the ultrasound diagnostic imaging apparatus 1 while examining a patient. Hence, the patient information displayed on the electric medical record terminal 3 is often information about the patient who undergoes the test. Therefore, by reading the patient information displayed on the electric medical record terminal 3 when registering patient information in the ultrasound diagnostic imaging apparatus 1, patient information registration therein can be easier.

When ending the patient information input processing, as shown in FIG. 5, the control unit 108 starts a scan (Step S102). More specifically, the control unit 108 performs transmission and reception of ultrasound with the transmission unit 102 and the reception unit 103 and generates ultrasound image data with the image generation unit 104 and the image processing unit 105. The control unit 108 stores the generated ultrasound image data frame by frame in the image memory unit 105a. At the time, an ultrasound diagnostic mode such as a B mode, a color Doppler mode, a pulse Doppler mode or an M mode and an ultrasound image display format such as a single mode in which one ultrasound image is displayed on one screen or a dual mode in which two ultrasound images are displayed on one screen may be set. The control unit 108 displays, on the display screen of the display unit 107a, the ultrasound diagnostic screen D1 on which an ultrasound image UD based on the ultrasound image data stored in the image memory unit 105a is displayed in the ultrasound image display region U1 as shown in FIG. 13. The control unit 108 repeats this action while the following steps are taken.

Next, the control unit 108 determines whether or not a setting change operation for any of various parameters is performed with the operation input unit 101 or the operation display unit 107 (Step S103).

When determining that a setting change operation for any of various parameters is performed (Step S103; YES), the control unit 108 changes the setting of a parameter(s) corresponding to an input to the operation input unit 101 or the operation display unit 107 (Step S104) and then moves to Step S103. Examples of the parameters include display depth and gain. The control unit 108 stores the parameter(s), the setting of which has been changed, into the RAM or the like.

When determining at Step S103 that no setting change operation for any of various parameters is performed (Step S103; NO), the control unit 108 determines whether or not to end the scan (Step S105). More specifically, the control unit 108 determines whether or not an end operation to end one test is received through the operation input unit 101. Instead of determining whether or not the end operation is received, the control unit 108 may determine whether or not patient information about another patient is set.

When determining not to end the scan (Step S105; NO), the control unit 108 determines whether or not a freeze operation is performed with the operation input unit 101 or the operation display unit 107 (Step S106). More specifically, the control unit 108 determines whether or not an operation to change the ultrasound image displayed as a moving image on the display unit 107a to a still image is performed. The freeze operation can be realized, as shown in FIG. 13, by a touch operation on the freeze button B1 while the ultrasound image is displayed as a moving image, for example.

When determining that no freeze operation is performed (Step S106; NO), the control unit 108 moves to Step S103. On the other hand, when determining that a freeze operation is performed (Step S106; YES), the control unit 108 performs freeze control to fix the ultrasound image which is displayed on the display screen 107a when the control unit 108 receives the freeze operation (Step S107). In the embodiment, the scan stops under the freeze control. However, the scan may continue with the ultrasound image fixed on the display screen.

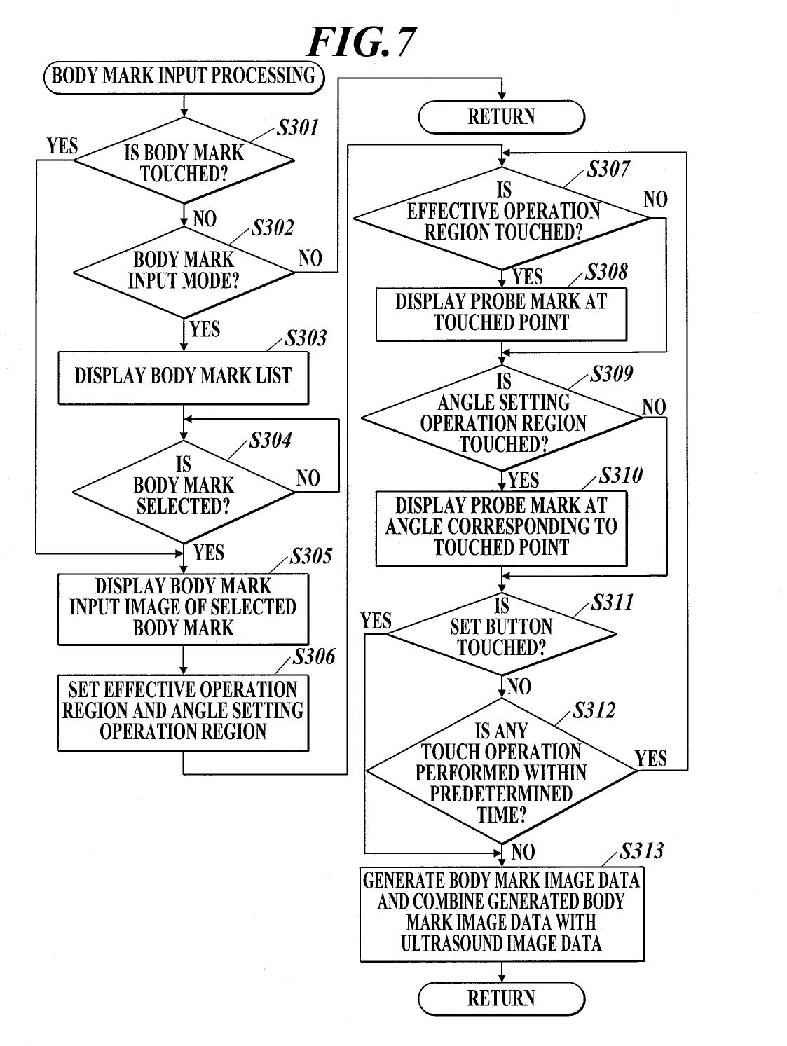

Next, the control unit 108 performs the body mark input processing (Step S108). A body mark is a simple recognition mark attached to an ultrasound image in such a way as to overlap the ultrasound image. With a body mark, when an ultrasound image is looked at, it can be visually recognized for which body part of a subject a test has been carried out. The body mark input processing is described with reference to FIG. 7.

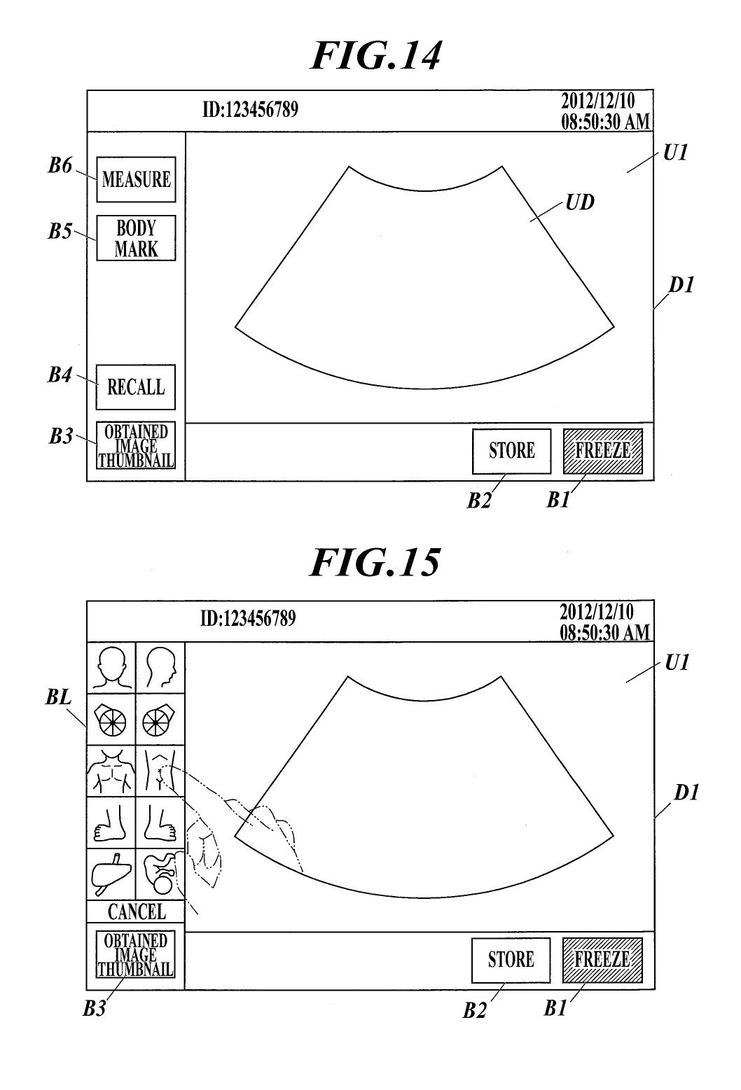

First, the control unit 108 determines whether or not a touch operation is performed on a body mark which is set as described below and displayed in such a way as to overlap the ultrasound image UD (Step S301). When determining that no touch operation is performed on the body mark (Step S301; NO), the control unit 108 determines whether or not to be in a body mark input mode (Step S302). More specifically, the control unit 108 determines whether or not to be in a body mark input mode on the basis of whether or not a touch operation on the body mark button B5 shown in FIG. 14 is detected.

When determining to be in a body mark input mode (Step S302; YES), the control unit 108 displays a body mark list BL on the left of the ultrasound image display region U1 as shown in FIG. 15 (Step S303). In the body mark list BL, a plurality of simple icons (body marks) representing body parts are displayed. Types of body marks can be appropriately set, and the number of types of body marks may be one. When determining not to be in a body mark input mode (Step S302; NO), the control unit 108 ends the body mark input processing.

Next, the control unit 108 determines whether or not a body mark is selected (Step S304). More specifically, the control unit 108 determines whether or not a body mark is selected on the basis of whether or not a touch operation is performed on any of the icons displayed in the body mark list BL as shown in FIG. 15.

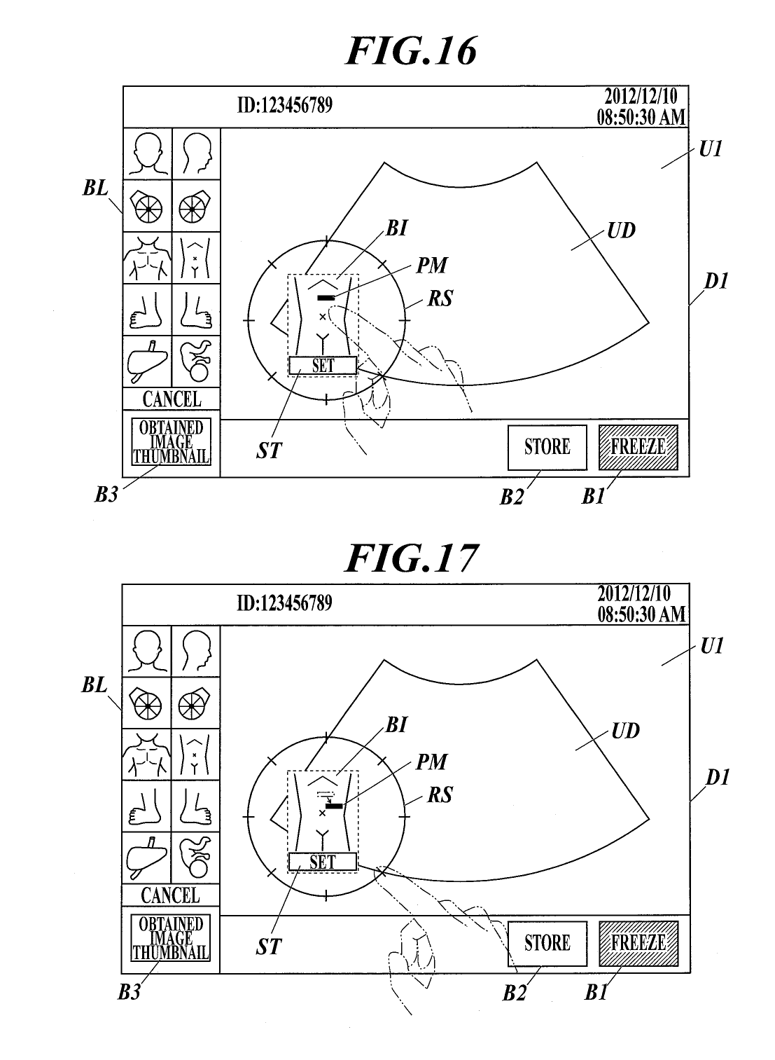

When determining that no body mark is selected (Step S304; NO), the control unit 108 repeats Step S304 until determining that a body mark is selected. On the other hand, when determining that a body mark is selected (Step S304; YES), the control unit 108 displays, as shown in FIG. 16, a body mark input image BI corresponding to the selected body mark with a probe mark PM, an angle setting guide RS and a set button. ST in such a way as to overlap the ultrasound image UD (Step S305).

The control unit 108 sets a region where the body mark input image BI is displayed as an effective operation region and sets a region where the angle setting guide RS is displayed as an angle setting operation region (Step S306). The effective operation region and the angle setting operation region are examples of operation receivable regions. The control unit 108 also sets a region where the set button ST is displayed as a set operation region (Step S306). Thus, the control unit 108 can receive touch operations on the body mark input image BI, the angle setting guide RS and the set button ST.

The control unit 108 determines whether or not a touch operation is performed on the effective operation region (Step S307). When determining that a touch operation is performed on the effective operation region (Step S307; YES), the control unit 108 displays the probe mark PM at the touched point (Step S308). More specifically, when a touch operation is performed on a certain point in the region where the body mark input image BI is displayed as shown in FIG. 16, the control unit 108 performs control to move the displayed probe mark PM to the touched point as shown in FIG. 17. Thus, the display position of the probe mark PM can be set at an appropriate point with one touch and intuitively. On the other hand, when determining that no touch operation is performed on the effective operation region (Step S307; NO), the control unit 108 moves to Step S309, skipping Step S308.

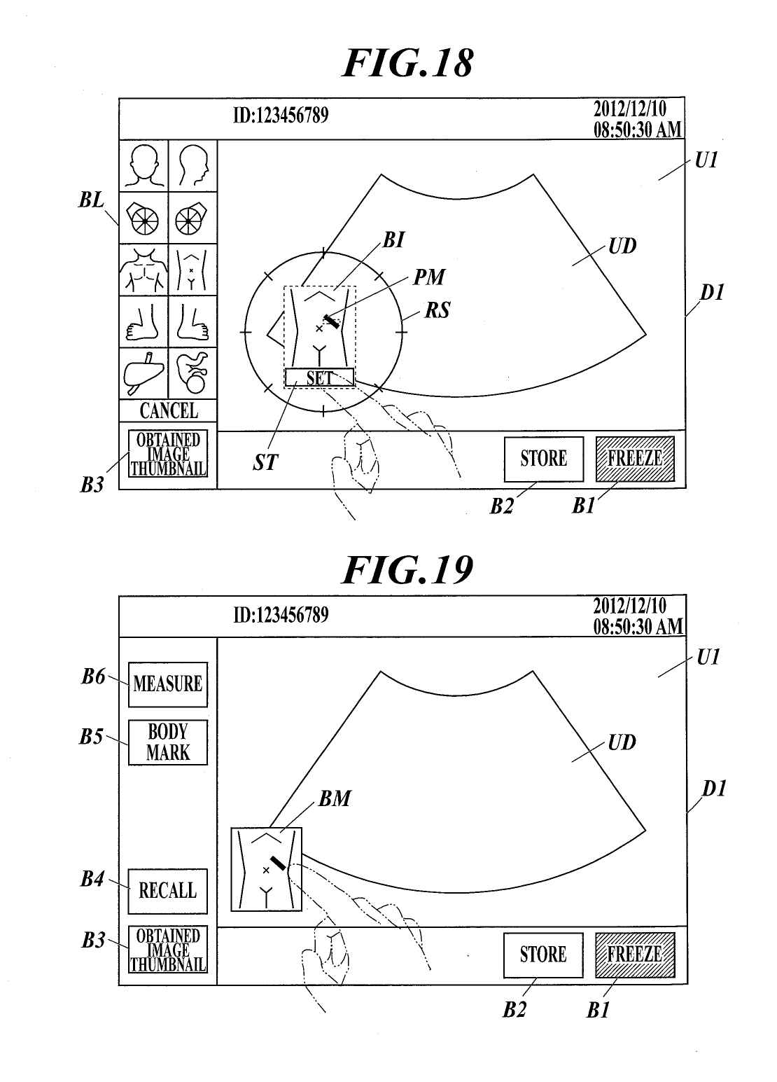

The control unit 108 determines whether or not a touch operation is performed on the angle setting operation region (Step S309). When determining that a touch operation is performed on the angle setting operation region (Step S309; YES), the control unit 108 displays the probe mark PM at an angle corresponding to the touched point (Step S310). More specifically, when a touch operation is performed on a certain point in the region where the angle setting guide RS is displayed as shown in FIG. 17, the control unit 108 performs control to display the probe mark PM at an angle corresponding to the touched point as shown in FIG. 18. Thus, the display angle of the probe mark PM can be appropriately set with one touch and intuitively. In the embodiment, the angle setting guide RS is composed of a circular image (ring-shaped image) enclosing the body mark input image BI, and hence a touch operation on the body mark input image BI and setting of the display angle of the probe mark PM can be performed without making a movement distance of a hand to perform the operations long. On the other hand, when determining that no touch operation is performed on the angle setting operation region (Step S309; NO), the control unit 108 moves to Step S311, skipping Step S310.

The control unit 108 determines whether or not a touch operation is performed on the set button ST (set operation region) (Step S311). When determining that no touch operation is performed on the set button ST (Step S311; NO), the control unit 108 determines whether or not any touch operation is performed within a predetermined time after the last touch operation (Step S312). In the embodiment, each time a touch operation is performed, the control unit 108 measures the elapsed time since the touch operation and determines whether or not the elapsed time reaches a predetermined time (for example, 30 seconds).

When determining at Step S311 that a touch operation is performed on the set button ST (Step S311; YES), namely, when detecting a touch operation on the set button ST performed as shown in FIG. 18 (Step S311; YES), or when determining at Step S312 that no touch operation is performed within a predetermined time after the last touch operation (Step S312; NO), the control unit 108 performs processing to generate body mark image data and combine the generated body mark image data with the ultrasound image data to set a body mark (Step S313) and then ends the body mark input processing. More specifically, the control unit 108 first performs processing to reduce image data of the body mark input image BI with the probe mark PM placed thereon to generate body mark image data. Then, the control unit 108 performs processing to combine the body mark image data with the ultrasound image data in such a way that the body mark image data overlaps the ultrasound image data. On the basis of the combined data, the control unit 108 displays the ultrasound image UD overlapped by the body mark BM in the ultrasound image display region U1 as shown in FIG. 19. At the time, the region where the body mark BM is displayed is set as a reset operation region, and a touch operation on the reset operation region is receivable. In the embodiment, the set button ST is arranged under the body mark input image BI to be adjacent thereto. Consequently, the display position of the probe mark PM can be set without making the movement distance of a hand long. Further, in the embodiment, a body mark is set when no touch operation is performed for a predetermined time. Consequently, the number of operations can be reduced, and the usability is improved.

When determining at Step S312 that a touch operation is performed within a predetermined time after the last touch operation (Step S312; YES), the control unit 108 moves to Step S307.

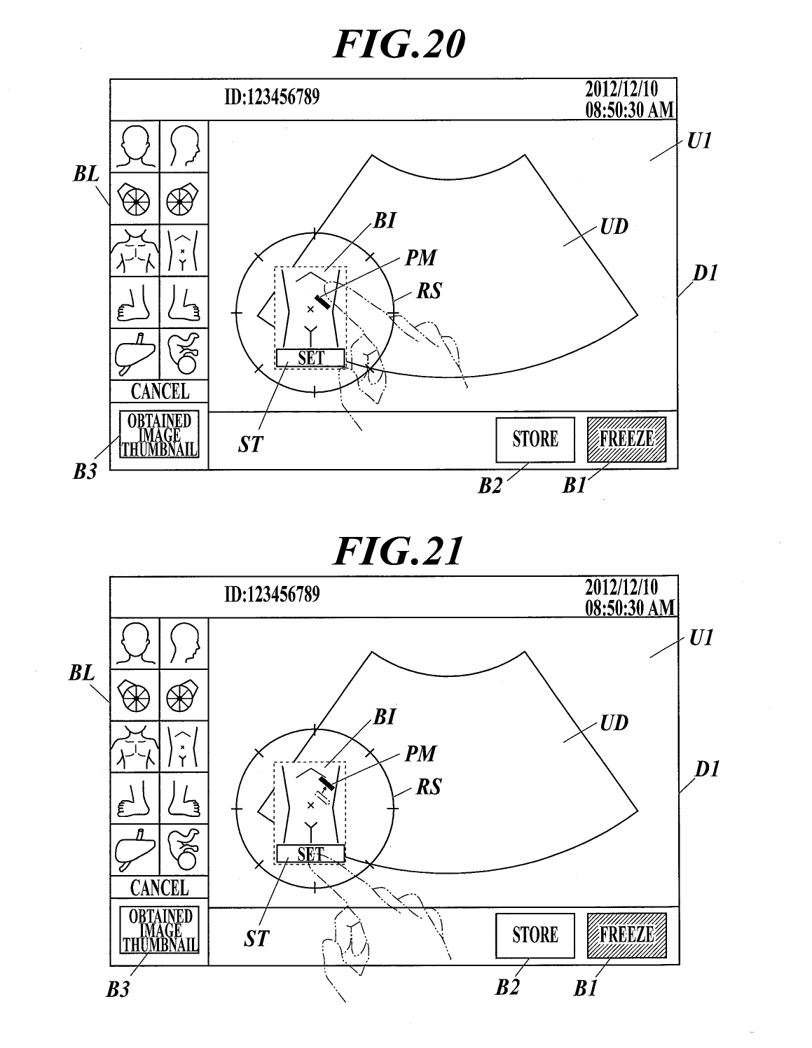

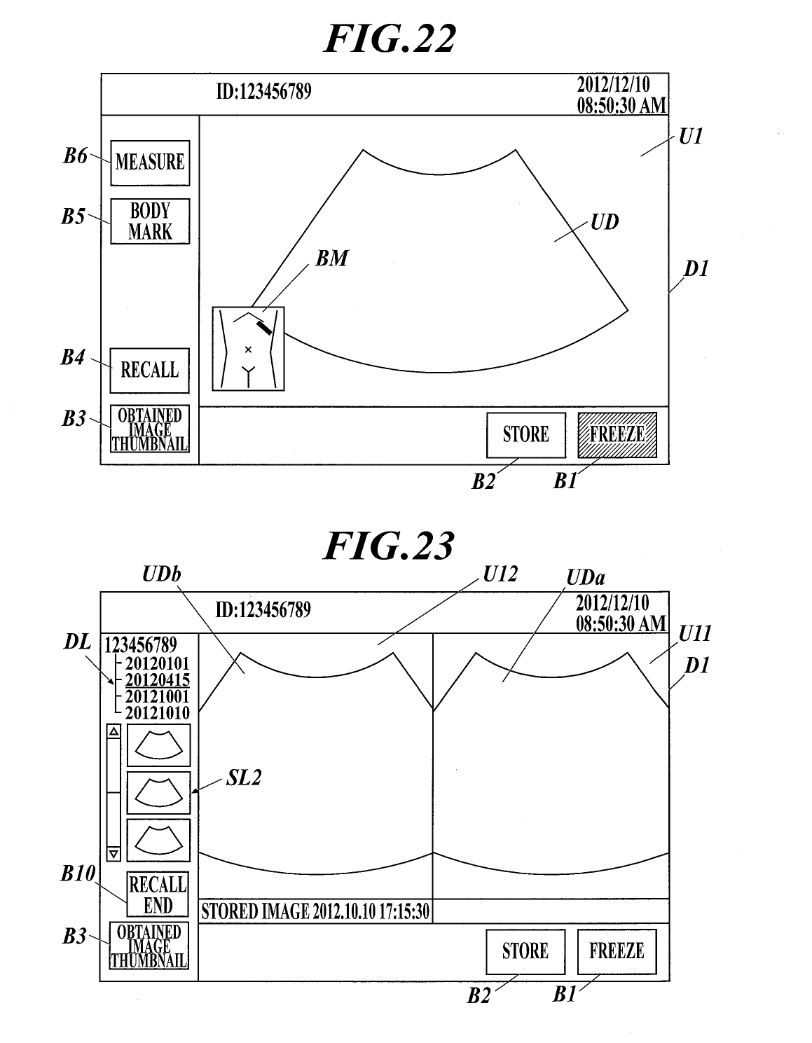

When determining at Step S301 that a touch operation is performed on the body mark (Step S301; YES), the control unit 108 moves to Step S305, skipping Steps S302 to S304, and takes Step S305 and the following steps. More specifically, for example, when a touch operation is performed on the body mark BM (reset operation region) as shown in FIG. 19, the body mark input image BI corresponding to the type of the body mark BM is displayed in such a way as to overlap the ultrasound image UD as shown in FIG. 20. At the time, the probe mark PM is displayed at the point and the angle set as described above. In this state, when a touch operation is performed on a certain point in the region where the body mark input image BI is displayed as shown in FIG. 20, the displayed probe mark PM is moved to the touched point as shown in FIG. 21. If a touch operation is performed on a certain point in the region where the angle setting guide RS is displayed too, the probe mark PM is displayed at an angle corresponding to the touched point in the above-described manner. When, as shown in FIG. 21, a touch operation is performed on the set button ST after the display position of the probe mark PM is changed, as shown in FIG. 22, the body mark BM is displayed with the probe mark PM displayed at the changed display position. According to the embodiment, with the configuration described above, the probe mark PM can be easily reset. That is, time and effort to search the body mark list BL for a desired body mark again to reset the probe mark PM can be saved, so that the usability is improved. Further, by making the display position of the body mark BM and the display position of the body mark input image BI close to each other, the operation regions to be in the body mark input mode, to set (determine) the display position of the probe mark PM and the like are concentrated in one small area, so that the movement of a hand to perform the operations can be small, and the usability is improved.

When ending the body mark input processing, as shown in FIG. 5, the control unit 108 determines whether or not an image store operation is performed (Step S109). More specifically, the control unit 108 determines whether or not an image store operation is performed on the basis of whether or not a touch operation is performed on the store button B2 displayed on the ultrasound diagnostic screen D1.

When determining that an image store operation is performed (Step S109; YES), the control unit 108 generates an image file on the basis of the combined data of the body mark image data and the ultrasound image data combined as described above (Step S110). More specifically, first, the control unit 108 reads the combined data from the image memory unit 105a and converts the read combined data into image data for storage (conversion-performed image data). The conversion-performed image data is, for example, bitmap image data. The conversion-performed image data may be image data compressed in a compression format such as JPEG (Joint Photographic Experts Group format). If no body mark is set, an image file is generated on the basis of the ultrasound image data, which is combined with no body mark image data. Next, the control unit 108 attaches the supplementary information to the conversion-performed image data to generate an image file composed of DICOM image data.

The control unit 108 stores the generated image file into the storage unit 109 (Step S111).

When determining at Step S109 that no image store operation is performed (Step S109; NO), the control unit 108 moves to Step S112, skipping Steps S110 and S111.

Then, the control unit 108 determines whether or not an unfreeze operation is performed (Step S112). More specifically, the control unit 108 determines whether or not an unfreeze operation is performed on the basis of whether or not a touch operation is performed on the freeze button B1 displayed on the ultrasound diagnostic screen D1 during the freeze control.

When determining that an unfreeze operation is performed (Step S112; YES), the control unit 108 performs unfreeze control to change the ultrasound image displayed as a still image on the display unit 107a to a moving image (Step S113) and moves to Step S102. On the other hand, when determining that no unfreeze operation is performed (Step S112; NO), the control unit 108 moves to Step S108.

When determining at Step S105 to end the scan (Step S105; YES) after generating the image file as described above, the control unit 108 ends the image file generation processing.

In a conventional ultrasound diagnostic imaging apparatus, for example, the position of a probe mark is set by operating a trackball or the like arranged on an operation input unit, the angle of the probe mark is set by rotating a dial switch or the like, and the position (and the angle) of the probe mark is determined by pressing a set switch, so that the movement distance of a hand in setting the probe mark is long depending on where the switches and the like are arranged. On the other hand, in the embodiment of the present invention, as described above, these functions are realized by the touch panel 107b, so that the body mark input image BI, the angle setting guide RS and the set button ST can be easily arranged close to each other. Consequently, the movement distance of a hand in setting the probe mark PM can be short, and the usability is improved.

Further, in the embodiment, the body mark input image BI displayed when the position of the probe mark PM is set is larger than the image of the body mark BM. Consequently, the operation in setting the position of the probe mark PM is easy.

In the embodiment, if a touch operation is performed on the recall button B4 on the ultrasound diagnostic screen D1 shown in FIG. 13, the ultrasound image display region U1 is divided into two regions arranged side by side so that a current test image display region U11 and a previously-stored image display region U12 are arranged as shown in FIG. 23. In the current test image display region U11, an ultrasound image UDa currently obtained is displayed. On the left of the previously-stored image display region U12, a date list DL of dates of the past tests with the ultrasound diagnostic imaging apparatus 1 which a patient has undergone is displayed. When a touch operation is performed on one of the dates displayed in the date list DL, thumbnails based on ultrasound image data stored on the date are displayed in a thumbnail list SL2. If a touch operation is performed on one of the thumbnails displayed in the thumbnail list SL2, ultrasound image data of the touched thumbnail is read from the storage unit 109, and an ultrasound image UDb based on the read ultrasound image data is displayed in the previously-stored image display region U12. That is, the ultrasound image UDa currently obtained and the ultrasound image UDb previously obtained are displayed side by side. If a touch operation is performed on a recall end button B10 in this state, the two regions (U11 and U12) into which the ultrasound image display region U1 are divided are hidden. If the ultrasound image data previously stored is moving image data, the ultrasound image UDb as a moving image can be displayed in the previously-stored image display region U12 on the basis of the ultrasound image data. According to the embodiment, for example, while a test is carried out, an ultrasound image at a past test can be easily referred to.

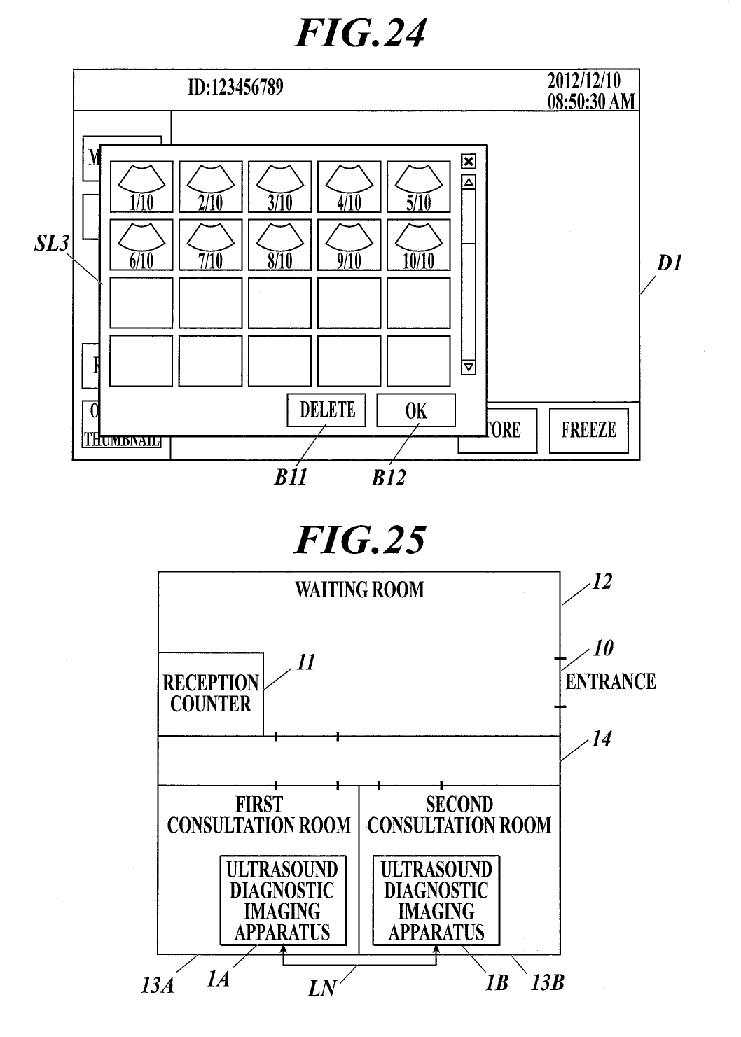

Further, in the embodiment, for example, if a touch operation is performed on the obtained image thumbnail button B3 after a test, as shown in FIG. 24, thumbnails based on the stored ultrasound image data of a patient having undergone the test are displayed in a thumbnail list SL3. With the thumbnail list SL3, for example, whether there is any body part which is a target for diagnosis (test) but the ultrasound image of which has not been obtained yet can be checked. If the thumbnail list SL3 shows an ultrasound image(s) desired to be deleted, the ultrasound image can be deleted by performing a touch operation on the thumbnail thereof and then performing a touch operation on a delete button B11. Further, an ultrasound image desired to be displayed in the ultrasound image display region U1 can be displayed therein by performing a touch operation on the thumbnail thereof displayed in the thumbnail list SL3 and then performing a touch operation on an OK button B12.

Further, in the embodiment, measurement about a subject can be performed by performing a touch operation on the measure button B6 with an ultrasound image of the subject displayed.

As described above, according to the embodiment, the touch panel 107b is disposed in such a way as to be superposed on the display screen of the display unit 107a. The control unit 108 displays a body mark representing a body part of a subject on the display screen of the display unit 107a. In addition, the control unit 108 sets the operation receivable region on the touch panel 107b in such a way as to be suitable for the displayed body mark, detects a touched point in the operation receivable region and performs display position setting to control the display unit 107a to display a probe mark representing the ultrasound probe 1b in form according to the detected touched point in the operation receivable region. Consequently, the display position of the probe mark PM can be set with one touch and accordingly can be set intuitively and easily, and the usability in inputting a body mark is improved. This is realized by the touch panel 107b, so that switches and the like are unneeded, and accordingly the ultrasound diagnostic imaging apparatus 1 can be smaller.

Further, according to the embodiment, in the display position setting, the control unit 108 controls the display unit 107a to display the probe mark at the detected touched point in the effective operation region. Consequently, the display position of the probe mark can be set with one touch and accordingly can be set intuitively and easily.

Further, according to the embodiment, in the display position setting, the control unit 108 controls the display unit 107a to display the probe mark at the display angle corresponding to the detected touched point in the angle setting operation region. Consequently, the display angle of the probe mark can be set with one touch and accordingly can be set intuitively and easily.

Further, according to the embodiment, the control unit 108 performs control to complete the display position setting when detecting a touch on the set operation region set in such a way as to be superposed on a set button displayed near the body mark. Consequently, an input operation(s) to the body mark and a completion operation to complete the display position setting can be performed within a small area, so that the movement distance of a hand between the input operation to the body mark and the completion operation can be short, and the usability is improved.

Further, according to the embodiment, in the display position setting, the control unit 108 controls the display unit 107a to display the body mark larger than the body mark which is displayed with the ultrasound image when the display position setting is completed. Consequently, the operation in setting the position of the probe mark is easy.

Further, according to the embodiment, in the display position setting, the control unit 108 performs control to complete the display position setting when detecting no touch on the touch panel 107b for a predetermined time. Consequently, the number of operations to complete setting the position of the probe mark can be reduced.

Further, according to the embodiment, the control unit 108 sets the reset operation region on the touch panel 107b in such a way as to be superposed on the body mark which is displayed with the ultrasound image when the display position setting is completed and performs the display position setting when detecting a touch on the reset operation region. Consequently, the display position of the probe mark can be reset intuitively and easily.

The embodiment of the present invention is an example of the medical image management system and the ultrasound diagnostic imaging apparatus of the present invention, and hence the present invention is not limited to the embodiment. Further, the detailed configurations and actions of the functional units of the medical image management system and the ultrasound diagnostic imaging apparatus are appropriately modifiable.

Further, in the embodiment, it is possible that the operation input unit 101 or the like receives input of text information as needed, and text image data based on the input and ultrasound image data are combined so that the text information is displayed with an ultrasound image(s).

Further, in the embodiment, an image file is generated on the basis of image data of an ultrasound image(s) as a still image(s), and the generated image file is stored. However, it is possible that image data of an ultrasound image(s) as a moving image(s) is obtained, and an image file is generated on the basis of the image data, and the generated image file is stored.

Further, in the embodiment, the display position and the display angle of the probe mark PM are set with touch operations on the effective operation region and the angle setting operation region, respectively. However, the display position and the display angle of the probe mark PM may be set with drag operations.

Further, in the embodiment, the set button ST is realized by the touch panel 107b. However, the set button ST may be provided on the operation input unit 101 or each of the operation input unit 101 and the touch panel 107a.

Further, in the embodiment, the body mark BM displayed when the display position setting to set the display position of the probe mark PM is completed and the body mark input image BI displayed when the body mark input mode is on may be the same size.

Further, in the embodiment, a hard disk, a nonvolatile semiconductor memory or the like is used as a computer readable storage medium of the programs of the present invention. However, this is not a limitation, and hence, for example, a portable storage medium such as a CD-ROM is usable as the computer readable storage medium. Further, a carrier wave is usable as a medium to provide data of the programs via a communication line.

Further, in the embodiment, as described above, one ultrasound diagnostic imaging apparatus 1 is arranged in one consultation room, and this ultrasound diagnostic imaging apparatus 1 is connected onto the network of the medical image management system 1000. However, as shown in FIG. 25, there is a case where two ultrasound diagnostic imaging apparatuses are arranged in two consultation rooms (a first consultation room 13A and a second consultation room 13B), respectively, and they are separated from a network of a medical image management system. At a small-sized medical facility such as an obstetrical and gynecological clinic, an ultrasound diagnostic imaging apparatus is used each time a doctor examines a patient, so that such arrangement may be used.

More specifically, for example, the first consultation room 13A and the second consultation room 13B face the waiting room 12 across the passage 14. In the first consultation room 13A and the second consultation room 13B, an ultrasound diagnostic imaging apparatus 1A and an ultrasound diagnostic imaging apparatus 1B are arranged, respectively. In this case, ultrasound image data obtained with the ultrasound diagnostic imaging apparatus 1A and ultrasound image data obtained with the ultrasound diagnostic imaging apparatus 1B are stored only in the first consultation room 13A and the second consultation room 13B, respectively.

In this arrangement, for example, there is a case where a patient has undergone a test with the ultrasound diagnostic imaging apparatus 1A in the first consultation room 13A previously and undergoes a test with the ultrasound diagnostic imaging apparatus 1B in the second consultation room 13B this time. In this case, because the two ultrasound diagnostic imaging apparatuses 1A and 1B are independent from the network of the medical image management system, a doctor can originally refer to patient information and ultrasound image data only through the ultrasound diagnostic imaging apparatus 1A or 1B with which the patient information and the ultrasound image data have been obtained. For example, the patient information and the ultrasound image data stored in the ultrasound diagnostic imaging apparatus 1A cannot be referred to through the ultrasound diagnostic imaging apparatus 1B.

Hence, in the case shown in FIG. 25, the two ultrasound diagnostic imaging apparatuses 1A and 1B are connected with each other via a local network LN. Then, for example, if a patient having undergone a test with the ultrasound diagnostic imaging apparatus 1A in the first consultation room 13A previously undergoes a test with the ultrasound diagnostic imaging apparatus 1B in the second consultation room 13B this time, the patient registration screen D2 is displayed on the display screen of the display unit 107a of the ultrasound diagnostic imaging apparatus 1B, and when a patient ID of the patient is input as described above, the ultrasound diagnostic imaging apparatus 1B searches the storage unit 109 for patient information agreeing with the input patient ID by using the patient ID as a search key. The ultrasound diagnostic imaging apparatus 1B also sends the patient ID to the ultrasound diagnostic imaging apparatus 1A connected via the local network LN to request for the patient information. The ultrasound diagnostic imaging apparatus 1A which receives the request searches the storage unit 109 for the patient information agreeing with the received patient ID by using the patient ID as a search key. If, as a result of the search, the ultrasound diagnostic imaging apparatus 1A stores therein the patient information and ultrasound image data previously obtained, the ultrasound diagnostic imaging apparatus 1A sends these to the ultrasound diagnostic imaging apparatus 1B. Thus, with the ultrasound diagnostic imaging apparatus 1B arranged in the second consultation room 13B, both the ultrasound diagnostic imaging apparatuses 1A and 1B can be searched for the patient information. That is, with the configuration described above, no matter which one of the two ultrasound diagnostic imaging apparatuses 1A and 1B is used to carry out a test on a patient, both the ultrasound diagnostic imaging apparatuses 1A and 1B can be searched for patient information about the patient. Consequently, convenience is improved.

In the above, two ultrasound diagnostic imaging apparatuses are connected with each other via a local network. However, the present invention can also be realized by three or more ultrasound diagnostic imaging apparatuses.

According to the configuration described above, it is enough for the ultrasound diagnostic imaging apparatuses to be communicable within a local network. Hence, it is unnecessary for each ultrasound diagnostic imaging apparatus to conform to a communication standard such as DICOM or to be provided with an external DB such as PACS. Consequently, the present invention can be realized at low cost.

This application is based upon and claims the benefit of priority under 35 USC 119 of Japanese Patent Application No. 2013-020000 filed on Feb. 5, 2013, the entire disclosure of which, including the description, claims, drawings and abstract, is incorporated herein by reference in its entirety.

What is claimed is:

1. An ultrasound diagnostic imaging apparatus comprising:
an ultrasound probe which outputs transmissive ultrasound to a subject and receives reflected ultrasound from the subject to obtain a reception signal;
a display unit including a display screen on which an ultrasound image based on ultrasound image data generated on the basis of the reception signal obtained by the ultrasound probe is displayed;
a touch panel disposed in such a way as to be superposed on the display screen of the display unit; and
a control unit which (i) displays a body mark representing a body part of the subject on the display screen of the display unit, (ii) sets an operation receivable region on the touch panel in such a way as to be suitable for the displayed body mark, (iii) detects a touched point in the operation receivable region and (iv) performs display position setting to control the display unit to display a probe mark representing the ultrasound probe in form according to the detected touched point in the operation receivable region.

2. The ultrasound diagnostic imaging apparatus according to claim 1, wherein
the operation receivable region includes an effective operation region set in such a way as to be superposed on the displayed body mark, and
in the display position setting, the control unit controls the display unit to display the probe mark at the detected touched point in the effective operation region.

3. The ultrasound diagnostic imaging apparatus according to claim 1, wherein
the operation receivable region includes an angle setting operation region set in such a way as to be superposed on an angle setting guide displayed around the body mark, and
in the display position setting, the control unit controls the display unit to display the probe mark at a display angle corresponding to the detected touched point in the angle setting operation region.

4. The ultrasound diagnostic imaging apparatus according to claim 1, wherein the control unit (i) sets a set operation region in such a way as to be superposed on a set button displayed near the body mark and (ii) in the display position setting, performs control to complete the display position setting when detecting a touch on the set operation region.

5. The ultrasound diagnostic imaging apparatus according to claim 1, wherein, in the display position setting, the control unit controls the display unit to display the body mark larger than the body mark which is displayed with the ultrasound image when the display position setting is completed.

6. The ultrasound diagnostic imaging apparatus according to claim 1, wherein, in the display position setting, the control unit performs control to complete the display position setting when detecting no touch on the touch panel for a predetermined time.

7. The ultrasound diagnostic imaging apparatus according to claim 1, wherein the control unit (i) sets a reset operation region on the touch panel in such a way as to be superposed on the body mark which is displayed with the ultrasound image when the display position setting is completed and (ii) performs the display position setting when detecting a touch on the reset operation region.
